(19) United States
(12) Patent Application Publication
(10) Pub. No.: US 2008/0082152 A1
(43) Pub. Date: Apr. 3, 2008

(54) Method of designing a multi-channel micro-electrical stimulation with an equivalent current source that works with an adiabatic baseband based on direct-carrier energy extraction

(75) Inventors: Cihun-Siyong (Alex) Gong, Kaohsiung (TW); Muh-Tian Shiue, Hsinchu City (TW); Chun-Hsien Su, Kaohsiung City (TW); Yin Chang, Taipei City (TW)

Correspondence Address: NIKOLAI & MERSEREAU, P.A., 900 SECOND AVENUE SOUTH, SUITE 820, MINNEAPOLIS, MN, 55402, US

(73) Assignee: National Central University, Jhongli City (TW)

(21) Appl. No.: 11/541,870

(22) Filed: Oct. 2, 2006

(51) Int. Cl. A61N 1/04

(52) U.S. Cl. 607/115

(57) ABSTRACT

A method of designing a multi-channel micro-electrical stimulation structure with an equivalent current source that works with an adiabatic baseband based on direct-carrier energy extraction matches with a baseband circuitry designed for energy recovery with the equivalent current source. A controllable pulse width modulation signal at the operating rate near to the carrier rate is used to implement the charge accumulation for the stimulation resolution on the time sequence axis. The design of equivalent current source may reduce the defects caused by a general D/A converter for full-scale stimulation resolution. Further, the adiabatic design is applied, so the redundant power consumption caused at the higher clock rate is minimized and, due to the switching current in the baseband circuitry significantly decreasing, only one voltage regulator circuit is required for the operation of analog and digital circuits that is imbedded into a chip, thereby more design cost being reduced.

### BACKGROUND OF THE INVENTION

1. Field of the Invention

This invention relates to a method of designing a multi-channel micro-electrical stimulation with an equivalent current source that works with an adiabatic baseband based on direct-carrier energy extraction and particularly to a method of effectively reducing the cost of design on a multi-channel micro-electrical stimulator.

2. Description of Related Art

Conventionally, a method of designing a digital-to-analog (D/A) converter is used to solve problems with a non-monotonic, rather non-linear error and a high design cost in a structure of high-resolution multi-channel micro-electrical stimulation.

In this invention, to solve the problems, the method of design of the equivalent current source is proposed that works with a method of time sequence analysis on cumulative electric charge with a pulse width modulation control signal for implementation of the stimulation effect equivalent to the conventional design.

Besides, for solving a problem with the decreasing of the number of channels that is caused by long stimulation time spent by a single channel after the time sequence analysis, the whole system is made to operate in a condition near to that of a carrier rate.

Since more power are consumed at the operating rate, the concept of adiabatic is proposed in this invention to implement the baseband circuitry for planning of the required pulse width modulation signal, thereby redundant power being eliminated for a solution to the previous defect of insufficient structure. The energy source of baseband circuitry is extracted from the carrier for not only the effect of synchronization but also the requirements of simplicity and saved area compared to that of the implementation in a method of design with a voltage-controlled oscillator. Further, the switching current of baseband circuitry significantly decreases. With the approach, only one voltage regulator circuit is required for the operation of analog and digital circuits that are imbedded into a chip, thereby more design cost being reduced.

Consequently, because of the technical defects of described above, the applicant keeps on carving unflaggingly through wholehearted experience and research to develop the present invention, which can effectively improve the defects described above.

### SUMMARY OF THE INVENTION

In order to solve the technical problems, in a conventional structure of multi-channel micro-electrical stimulation, a method of designing an analog-to-digital (A/D) converter is applied. However, there are problems with a non-monotonic, rather non-linear error, and a high design cost in such an approach.

In order to solve the technical problems, a method of designing a low-cost and high-performance multi-channel micro-electrical stimulation structure that works with an adiabatic baseband based on direct-carrier energy extraction is provided.

In this invention, a method of designing a multi-channel micro-electrical stimulation structure for full-level charge resolution that works with an adiabatic baseband based on direct-carrier energy extraction is provided to significantly reduce defects caused by a general A/D converter.

In this invention, the method of designing the multi-channel micro-electrical stimulation structure consuming redundant power generated at a higher clock rate and further minimized that works with an adiabatic baseband based on direct-carrier energy extraction is provided.

In order to achieve the objective mentioned above, the method of designing the multi-channel micro-electrical stimulation with an equivalent current source that works with an adiabatic baseband for direct-carrier energy extraction is proposed, comprising steps below:
a. a method of design of the equivalent current source is applied to implement a stimulation array in which each stimulation unit is provided with an equivalent constant current source;
b. the stimulation array carries out stimulation in each channel by a set of stimulation control signals so as to adjust the cumulative energy of stimulation charge by means of proper electrical stimulation strength through a time sequence axis;
c. a digital baseband circuitry being arranged at a demodulation receiving end, and after the modulation signal of carried stimulation control signal is demodulated, clock extraction being executed and the stimulation control signals being processed;
d. an adiabatic design is applied to significantly reduce the power required by the digital baseband circuitry in the aspect of switching current; and
e. a method of direct carrier being applied to extract the power required by the digital baseband circuitry, in which a carrier signal coupled into a receiver coil, through an input matching and a step voltage drop circuit, is fed into a pair of dual circuits to extract the complementary energies required by the subsequent baseband circuitry.

For a virtue compared with that of the prior art, the concept of energy recovery is proposed in this invention to implement the baseband circuitry for planning of the required pulse width modulation signal, thereby redundant power being eliminated for a solution to the previous defect of insufficient structure. The energy source of baseband circuitry is extracted from the carrier for not only the effect of synchronization but also the requirements of area simplification and saving and power saving compared to that of the implementation in a method of design with a voltage-controlled oscillator, thereby the defects caused by the general D/A converter applied to the micro-electrical stimulation being decreased.

### BRIEF DESCRIPTION OF THE DRAWINGS

FIG. 1 is a schematic view illustrating a micro-electrical stimulation array provided with an equivalent current source and its complete stimulation time sequence arrangement;

FIG. 2 is a schematic view illustrating an adiabatic unit designed;

FIG. 3 is a schematic view illustrating a circuit in which it extracts the internal operating clocks and the baseband energy directly from the coupled signal; and

FIG. 4 shows a simulation result given from the integral operation of a human body temperature variation (degree Celsius from 35 to 41) and related verification specifications for the same.

### DETAILED DESCRIPTION OF THE PREFERRED EMBODIMENTS

Now, the present invention will be described more specifically with reference to the following embodiments. It is to be noted that the following descriptions of preferred embodiments of this invention are presented herein for purpose of illustration and description only; it is not intended to be exhaustive or to be limited to the precise form disclosed.

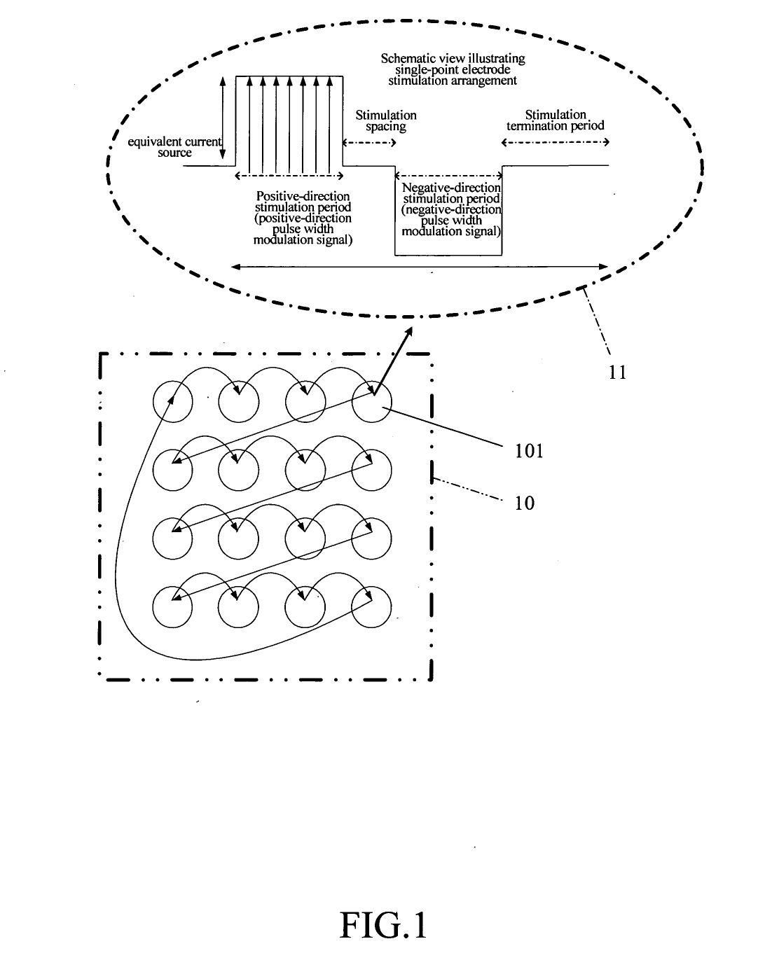

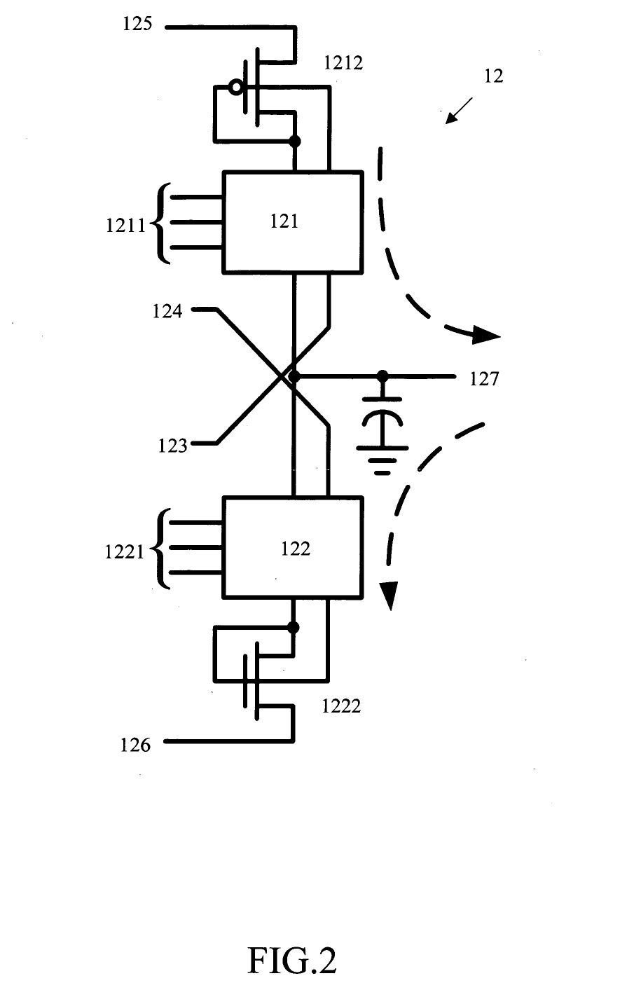

With reference to FIGS. 1 through 3, a method of designing a multi-channel micro-electrical stimulation with an equivalent current source that works with an adiabatic baseband based on direct carrier energy extraction is proposed in this invention, comprising steps below:
a. a method of current source is applied to implement an equivalent current-based stimulation array 10, as shown in FIG. 1, in which each stimulation unit 101 is provided with an equivalent constant current source, but neither a binary-weighted method applied for a general D/A converter nor a hybrid method of design for segment is not applied.
b. In order to make the stimulation normally proceed, the stimulation array 10 carries out stimulation in each channel through the stimulator by a set of stimulation control signals 11 so as to adjust the cumulative charge of stimulation by means of proper electrical stimulation strength through a time sequence axis for achievement of a equivalent stimulation effect, in which the stimulation control signals 11 may be a pulse width modulation signal.
c. In order to restore information received at an embedded receiving end to data transmitted externally and further arrange proper pulse width modulation signal, a digital baseband circuitry designed with adiabatic approach 12, as shown in FIG. 2, is provided at a receiving end of a modulation signal that carries stimulation control signals 11. And after the modulated signals are demodulated, clock extraction is executed and the stimulation control signals 11 are arranged. For solving a problem with the decreasing of the number of channels that is caused by long stimulation time spent by a single channel after the time sequence analysis, counting signals employed for arrangement of pulse width control must operate in a condition near to that of a carrier rate for supporting the micro-electrical stimulation system provided with equal number of channels.
d. The increasing of operation speed will cause the power consumption of digital baseband circuitry to significantly increase, so for the objective of design according to the requirement of power efficiency, an adiabatic design 12 is applied to significantly reduce the power required by the digital baseband circuitry in the aspect of switching current; thus, a higher internal operation speed may be available in an equivalent structure designed formerly or even in a condition of lower power consumption, in which the designed adiabatic structure 12 used in the digital baseband circuitry comprises a P-type transistor 121 and a N-type transistor 122 with the corresponding input terminals 1211 and 1221, respectively; the body of P-type transistor 121 is connected to a DC power supply 123, while the body of N-type transistor 122 is connected to a reference ground terminal 124; next, the P-type transistor 1212 is connected to adiabatic baseband power 1125, while the N-type transistor 1222 is connected to adiabatic baseband power 1126, and thereby the output signal of adiabatic circuit from the P-type transistor 121 and the N-type transistor 122 is 127.

Besides, the adiabatic design is applied because transistors in a general digital integrated circuit consume most of the energy caused by the switching current despite the effect of the leakage current.

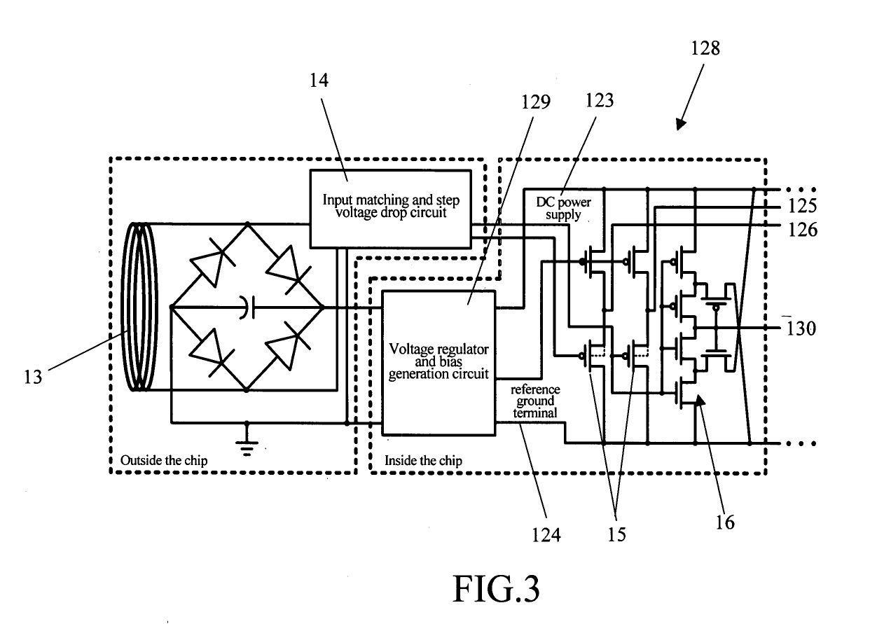

Since the transistors must charge the output point to a whole magnitude (or discharge it to a low potential) at an extremely short time in a general implementation, and the current extracted from a voltage supply terminal or an output terminal will certainly be enormous. For this reason, in the adiabatic design, progressive voltage supply and discharge to a low level are applied so that “momentary switching” does not exist and the current is made to decrease for achievement of low power consumption. It is nevertheless especially noted that although the saving of more power consumption, the drive capability is limited; thus a high-speed design is not available, it is quite applicable to a biomedical implant and wireless identification label Microsystems.
e. A set of particular power supply is required in the digital baseband circuitry designed for adiabatic circuit 12, as shown in FIG. 3, so a direct carrier extraction 128 is applied for the power of the digital baseband circuitry under different scenario of the digital binary modulations. A carrier signal coupled into a receiving end of coil 13, through an input matching and a step scale voltage drop circuit 14, is directly fed into a pair of dual circuits 15 to extract complementary energy 125 and 126 of the adiabatic baseband circuitry that are required. Schmidt trigger circuit 16 in the range of direct carrier extraction 128 extracts timing pulse signals 130 required by the adiabatic baseband circuitry 12. A voltage regulator and a bias generation circuit 129 supply power and bias voltage that are respectively required by the digital baseband circuitry and the multi-channel micro-electrical stimulation circuit.

Thus, in this invention, the method of design of the equivalent current source matches with the digital baseband circuitry designed in the concept of low power consumption based on energy recovery. The controllable pulse width modulation signal arranged at the operating rate near to the carrier rate is used to implement the electric charge accumulation for the stimulation resolution on the time sequence axis, for the purpose of achievement of equivalent electrical stimulation effect, thereby this invention making the cost down and the performance up.

Next, the method of design of the equivalent current source according to this invention may also significantly reduce the defects caused by the general D/A converter used in the micro-stimulation system for the full-level stimulation resolution.

Further, the adiabatic design is applied, so the redundant power consumption caused at the higher clock rate is minimized and, due to the switching current in the baseband circuitry that significantly decreases, only one voltage regulator circuit is required for the operation of analog and digital circuits that are imbedded into a chip. Thus, this invention may make the cost down and the resolution up.

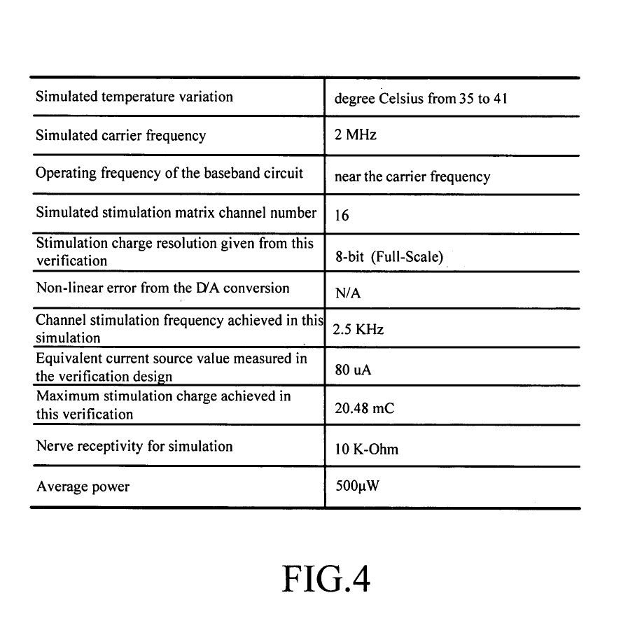

Further, FIG. 4 shows a simulation result given from the integral operation of a human body temperature variation (degree Celsius from 35 to 41) and related verification specifications for the same. A manufacturing process technology on 0.18 micron, single Poly, 6-layer Metal is applied for design and experiment, in which the proposed method may significantly reduce the defects caused by the general D/A converter used for a design work for the full-level stimulation resolution.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method of designing a multi-channel micro-electrical stimulation structure with an equivalent current source that works with an adiabatic baseband based on direct-carrier energy extraction, comprising steps below:
a. a method of design of the equivalent current source being applied to implement a stimulation array;
b. the stimulation array carrying out stimulation in each channel and giving a set of stimulation control signals so as to adjust the cumulative charge of stimulation by means of proper electrical stimulation strength through a time sequence axis;
c. a digital baseband circuitry being arranged at a demodulation signal receiving end, and after the modulation signal of carried stimulation control information is demodulated, clock extraction being executed and the stimulation control signals being processed;
d. an adiabatic design being applied to significantly reduce the power required by the digital baseband circuitry in the aspect of switching current, and a design cost of extra voltage regulator circuit being reduced; and
e. a method of direct carrier being applied to extract the power required by the digital baseband circuitry, in which a carrier signal coupled into a receiver coil, through an input matching and a step voltage drop circuit, is fed into a pair of dual circuits to extract the complementary energies required by the subsequent digital baseband circuitry.

2. The method of designing the multi-channel micro-electrical stimulation structure with the equivalent current source that works with the adiabatic baseband based on direct-carrier energy extraction according to claim 1, wherein each stimulation unit in the stimulation array at step a. is provided with an equivalent constant current source.

3. The method of designing the multi-channel micro-electrical stimulation structure with the equivalent current source that works with the adiabatic baseband based on direct-carrier energy extraction according to claim 1, wherein the stimulation control signals are utilized to generate the pulse width modulation control signal.
